(19) United States
(12) Patent Application Publication
(10) Pub. No.: US 2006/0178692 A1
(43) Pub. Date: Aug. 10, 2006

(54) Dilator for body passageway

(76) Inventors: Alexander Condrea, Haifa (IL); Aric Vadasz, Haifa (IL)

Correspondence Address: NATH & ASSOCIATES, 112 South West Street, Alexandria, VA, 22314, US

(21) Appl. No.: 10/514,543

(22) PCT Filed: May 15, 2003
(86) PCT No.: PCT/IL03/00400

§ 371 Date: Sep. 26, 2005

(30) Foreign Application Priority Data
May 15, 2002 (IL) 149687

(51) Int. Cl. A61M 29/00

(52) U.S. Cl. 606/192

(57) ABSTRACT

A device for dilating a body passageway. The device comprises an elongaed hollow shaft and a balloon at the distal end of the shaft in fluid communication with the lumen of the shaft, the balloon having at least a cylindrical portion. The balloon is formed from a flexible, unstretchable material capable of withstanding an inflation pressure of at least 10 bar. When the balloon in an uninflated state is collapsed onto the shaft the diameter of the balloon on the shaft is at most 35% of the diameter of the cylindrical portion in an inflated state.

### FIELD OF THE INVENTION

The present invention relates to medical devices and more specifically to such devices for dilating a body passageway.

### BACKGROUND OF THE INVENTION

It is often necessary to dilate a body passageway, such as a blood vessel, urethra, or cervix. In the case of the cervix, for example, it is sometimes necessary to dilate the cervix in order to allow access of surgical instruments into the uterine cavity during a medical procedure. Such procedures include induced abortion, completion of a spontaneous abortion, and operative hysteroscopy.

The human cervix is a tubular structure between 2 to 5 cm in length. The cervical canal is usually closed, but a catheter having a diameter of 2 to 3 mm can be introduced into the cervical canal with only minor discomfort to a female patient in the absence of any anesthesia. It is usually required to dilate the cervix to a diameter between 10-12 mm.

One known method for dilating the cervix uses Hegar dilators. These are essentially metal rods. A dilator of a relative small caliber is inserted into the cervix to achieve a small dilation. The rod is removed, and a rod of slightly larger caliber is inserted into the cervix. This rod is then removed, and the process repeated, each time with a rod of larger caliber, until the desired dilation has been achieved. This procedure requires some form of anesthesia, and may damage the cervix.

U.S. Pat. No. 4,624,258 to Stubbs discloses a hygrometric dilator comprising an insertion body containing the vegetative stalks of Laminaria japonica. The insertion body is inserted into the cervix, whereupon the Laminaria expands as it absorbs fluids present in the cervix, causing the cervix to dilate.

WO 81/01098 discloses a device for inflating a cervix comprising an inflatable latex balloon. The balloon is enclosed in a sleeve formed from an inelastic material in order to allow the balloon to withstand high pressures.

U.S. Pat. No. 4,137,222 to Leininger discloses an inflatable device having an inflatable balloon with an enlarged bulbous portion at one end. When the balloon is inflated, the enlarged portion expands inside the uterus while the remainder of the balloon inflates in the cervix. The enlarged portion prevents the balloon from being expelled out of the cervix.

### SUMMARY OF THE INVENTION

The present invention provides a device and method for dilating a body passageway. While the device will be described in relation to dilating a cervix, it should be understood that the device of the invention may be used to dilate other body cavities such as a urethra or blood vessel.

The device of the invention comprises a hollow shaft having a distal end and a proximal end. The shaft typically has a diameter of about 1 to 2 mm. At the distal end of the shaft is an inflatable balloon. A pressurized fluid is made to flow in the lumen of the hollow shaft from the proximal end to the distal end so as to inflate the balloon. The balloon has at least a portion that is cylindrical in shape when inflated.

The balloon is formed from a flexible essentially unstretchable material. The material of the balloon is further selected so that the inflated balloon can withstand pressures of up to 10 bar, more preferably 20 bar, and still more preferably, 30 bar. The wall of the balloon is sufficiently thin so that when the uninflated balloon is collapsed onto the shaft, the diameter of the balloon on the shaft is at most 35%, more preferably 30%, even more preferably 25% of the diameter of the inflated cylindrical portion of the balloon. The thickness of the wall of the balloon is preferably less than 0.1 mm, more preferably less than 0.08 mm, and still more preferably around 0.05 mm.

For example, the inventors have found that a cylindrical balloon having an inflated diameter of 12 mm formed from polyethylene perephthalate (PET) or polyamide and having a wall thickness of 0.05 mm can withstand pressures up to at least 14 bar. Since the maximum pressure that a cylindrical balloon can withstand is proportional to its wall thickness, a balloon of this material and inflated diameter having a wall thickness of 0.10 mm can withstand pressures up to 28 bar. Similarly, a balloon of this material and inflated diameter having a wall thickness of 0.20 mm can withstand pressures up to 42 bar. A cylindrical balloon having an inflated diameter of up to 10 mm and a wall thickness up to 0.2 mm, will have, when collapsed onto a 1 mm diameter shaft, a diameter less than 3 mm.

The balloon may optionally be formed so as to form a spherical bulb at the distal end of the inflated balloon having a diameter greater than that of the cylindrical portion. Alternatively, the device may comprise a cylindrical balloon and a separate spherical or ellipsoidal balloon. A distally located spherical balloon allows the cylindrical portion of the balloon to be properly positioned in the cervix, as described in detail below. Furthermore, a spherical balloon can withstand twice the pressure of a cylindrical balloon from the same material and wall thickness. Thus, in one embodiment, the device also includes a spherical balloon that is used, to expand a portion of the passageway.

The proximal end of the shaft is adapted to be connected to a source of a pressurized fluid. The fluid is preferably an incompressible fluid such as water or saline. The fluid may be delivered from the proximal end of the shaft to the distal end of the shaft and into the balloon by any means of pressurizing the fluid. For example, the fluid may be loaded into a syringe that is placed in fluid communication with the lumen of the shaft. The fluid is then manually pressurized by displacing the piston of the syringe.

In a preferred embodiment, the fluid is pressurized by an electrical pump that delivers the fluid through the shaft to the balloon at a predetermined flow rate. The flow rate is selected to be slow enough so as to inflate the balloon at a rate that does not cause severe pain to the patient. The device may thus be used without anesthetizing the patient.

Since the balloon is formed from an unstretchable material, when an incompressible fluid is used to inflate the balloon, the volume of the balloon may be determined at any time from the amount of fluid delivered to the balloon. In particular, the maximum volume of the balloon is obtained when that volume of fluid has been delivered to the balloon.

Thus, in its first aspect, the invention provides a device for dilating a body passageway comprising:

(a) an elongated hollow shaft having a lumen, a proximal end and a distal end, and

(b) a balloon at the distal end of the shaft in fluid communication with the lumen of the shaft, the balloon having at least a cylindrical portion, characterized in that:
(i) the balloon is formed from a flexible, unstretchable material capable of withstanding an inflation pressure of at least 10 bar.
(ii) when the balloon in an uninflated state is collapsed on the shaft, the diameter of the balloon on the shaft is at most 35% of the diameter of the cylindrical portion in an inflated state.

In its second aspect, the invention provides a method for dilating a body passageway comprising inserting the cylindrical portion of the balloon of a device in accordance with the invention into the passageway and inflating the balloon.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a dilator having a balloon, in accordance with one embodiment of the invention;

FIG. 2 shows the dilator of FIG. 1 with the balloon in an uninflated state after insertion into a cervix, FIG. 3 shows the dilator of FIG. 1 after insertion into a cervix with the balloon in a partially inflated state;

FIG. 4 shows the dilator of FIG. 1 after insertion into a cervix with the balloon in an inflated state;

FIG. 5 shows a dilator having two balloons in accordance with one embodiment of the invention;

FIG. 6 shows a dilator having two balloons and a shaft with two lumens in accordance with another embodiment of the invention.

FIG. 7 shows a dilator having two balloons in accordance with another embodiment of the invention; and

FIGS. 8 to 11 show deployment of the dilator of FIG. 7.

### DETAILED DESCRIPTION OF THE INVENTION

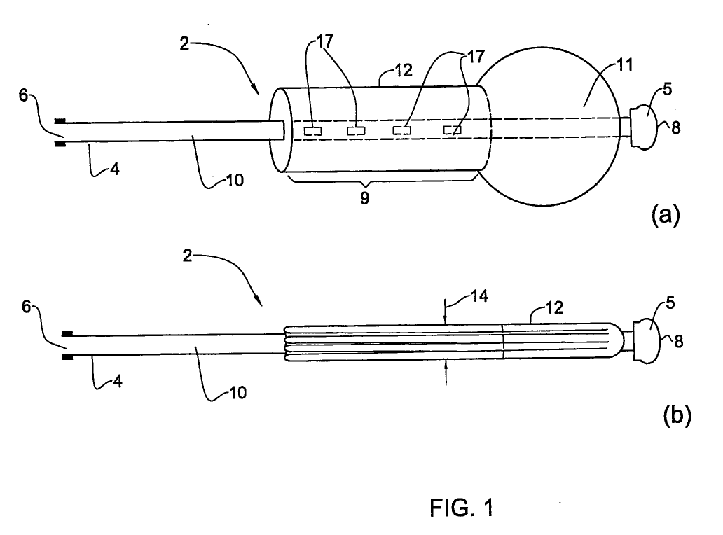

FIG. 1 shows a device for dilating a body passageway, in accordance with one embodiment of the invention. The device generally indicted by 2, includes an elongated shaft 4 having a proximal end 6 and a distal end 8. The shaft 4 has an outer diameter of 1 to 2 mm and encloses a lumen 10. The shaft is formed from a flexurally resilient material such as plastic, stainless steel, or polyurethane. A blunt cap 5 at the distal end 8 of the shaft 4 prevents damage to tissues during insertion.

An inflatable balloon 12 located at the distal end of the shaft 4 is formed from a flexible material that is essentially unstretchable. The balloon 12 is shown in FIG. 1a in its inflated state. The inflated balloon 12 has a generally cylindrical shaped body portion 9 with a length of about 6 cm and an inflated diameter of about 1.2 cm. The cylindrical portion is coaxial with the shaft and has an annular cross-section. The balloon terminates in an ellipsoidal portion 11 having an axis of about 15 mm perpendicular to the axis of the shaft 4.

FIG. 1b shows the device 2 with the balloon 12 in its uninflated state. The balloon 12 has been collapsed and wrapped around the shaft 4. The wall of the balloon 12 has a thickness of about 0.02-0.05 mm, so that the diameter 14 of the collapsed balloon on the shaft 11 does not exceed 3 mm.

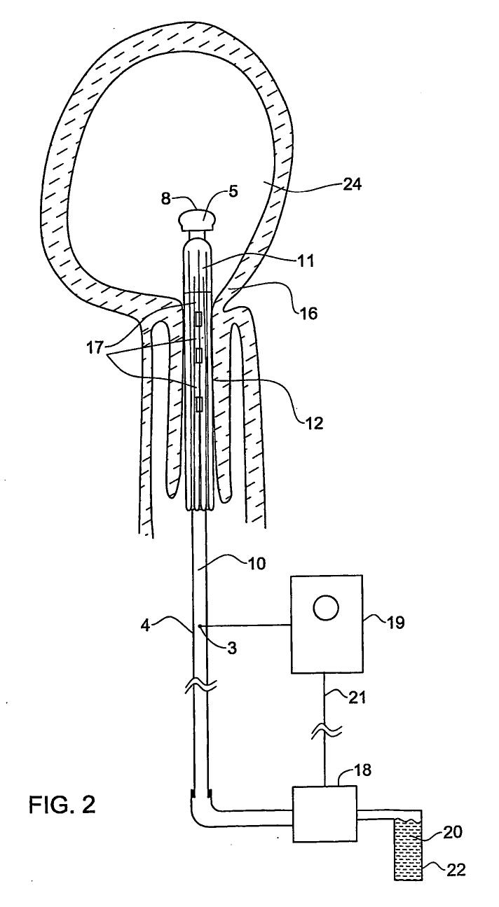

The balloon 12 is delivered to the passageway to be dilated with the uninflated balloon collapsed on the shaft 4. FIG. 2 shows the device 2 after delivery of the balloon 12 in its uninflated state to a cervix 16. The distal end 8 and the ellipsoidal portion 11 are located in the uterus 24. An electric pump 18 is used to deliver an incompressible fluid 20 in a reservoir 22 to the balloon 12. The fluid flows from the pump 18 through the lumen 10 of the shaft 4 through openings 17 in the distal end of the shaft 4 into the balloon 12. The pump 18 is may be programmable so as to deliver the fluid 20 at a rate that builds up pressure in the balloon 12 sufficiently slow so as to prevent excessive pain to the patient in the absence of anesthesia. Alternatively, the pump 18 may have a selectable flow rate by means of a flow rate selector 19. The flow rate selector 19 may be located remote from the pump 18, for example, by means of a cable 21, or by a remote control (not shown). A remotely positionable flow rate selector 19 may be controlled by the patient so that the patient may select a flow rate that does not cause excessive pain. The pump 18 may be connected to a pressure gauge 3 that measures the fluid pressure inside the balloon 12 during inflation. In this case, the pump 18 may be programmed to deliver fluid to the balloon 12 so as to obtain a predetermined rate of increase in pressure. The pump 18 may further be programmable to discontinue delivery of the fluid 20 to the balloon when the volume of fluid 20 delivered to the balloon 12 is equal to the maximum capacity of the balloon 4.

As the balloon 12 is inflated, the ellipsoidal portion 11 of the balloon inflates before the cylindrical portion 9, even though the thickness of the wall of the balloon is uniform throughout the cylindrical and ellipsoidal portions of the balloon 12.

This is because the ellipsoidal portion 11, being located in the uterus 24, is subjected to a lower external pressure than the cylindrical portion 11 in the cervix 16.

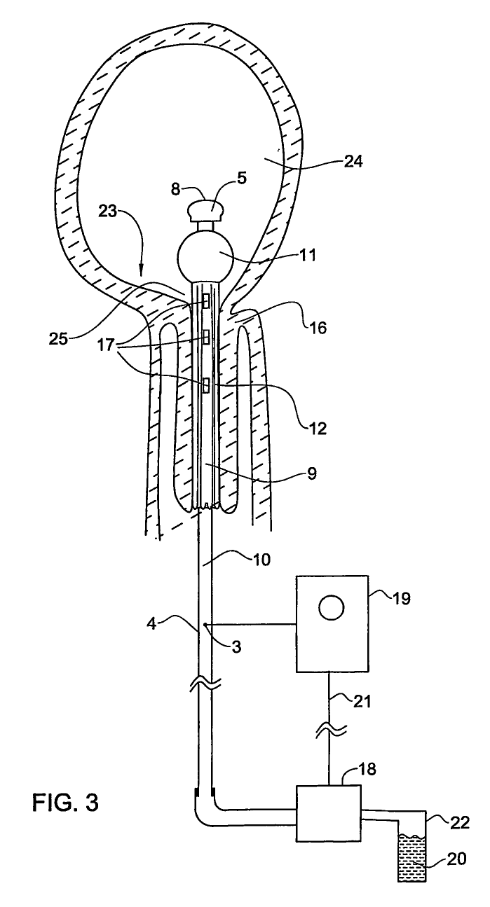

FIG. 3 shows the device 2 after partial inflation of the balloon 12, showing the ellipsoidal portion 11 is almost completely inflated, and the cylindrical portion 9 only partially inflated. At this point, the balloon 12 is moved proximally in the direction of the arrow 23, so as to lodge the ellipsoidal portion 11 in the internal os 25. This ensures that the cylindrical portion is appropriately positioned in the cervix. Inflation of the balloon 12 continues until the balloon 12 has attained its maximum volume, at which time the cylindrical portion 9 is completely distended and has a diameter of at least 10 cm and preferably 12 cm. The pressure in the balloon when dilation of cervix 16 is complete may exceed 10 bar, 20 bar, 30 bar or 40 bar. FIG. 4 shows the dilator 2 with the balloon 4 completely inflated and the ellipsoidal portion 11 of the balloon 4 lodged in the internal os 25. The balloon 12 is now deflated and the device 2 is withdrawn from the body.

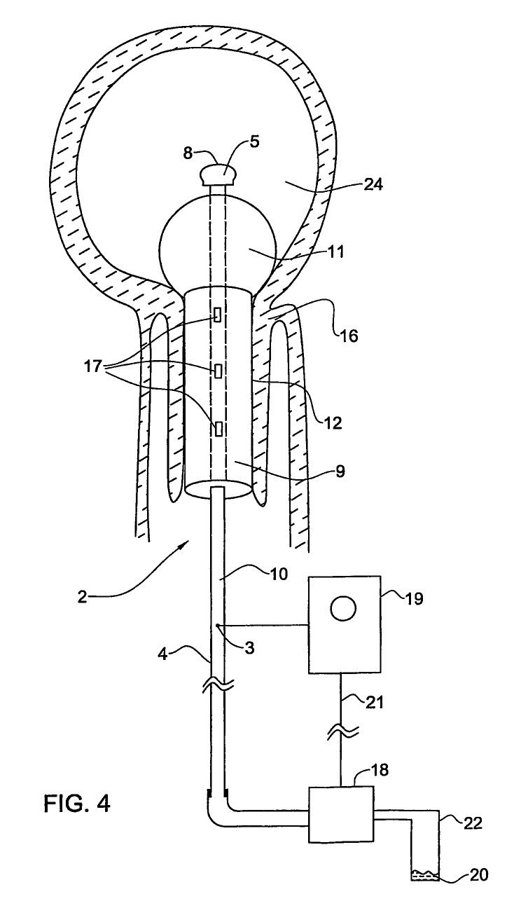

FIG. 5 shows another embodiment of the device of the invention, generally indicated by 35. The device 35 has features in common with the device 2, and similar components are identified with the same numeral without further comment. The embodiment 35 has two balloons 36 and 37, that are shown in their inflated state in FIG. 5. The balloon 36 has a cylindrical shape when inflated, and has dimensions similar to those of the cylindrical portion 9 of the balloon 12. The balloon 37 is ellipsoidal in shape and has dimensions similar to those of the ellipsoidal portion 11 of the balloon 12. The device 35 is used similarly to the device 2 as shown in FIGS. 2 to 4. Both balloons 36 and 37 are in fluid communication with the shaft 11. As explained above with reference to the ellipsoidal portion 11 of the balloon 12, the ellipsoidal balloon 37 will inflate before the cylindrical balloon 36. When the ellipsoidal balloon 37 is inflated, the shaft 4 is moved proximally until the ellipsoidal balloon 37 is lodged in the internal os. The shaft 4 is then moved distally a distance about equal to the spacing between the two balloons 36 and 37 along the shaft, so as to position the cylindrical balloon 36 in the cervix.

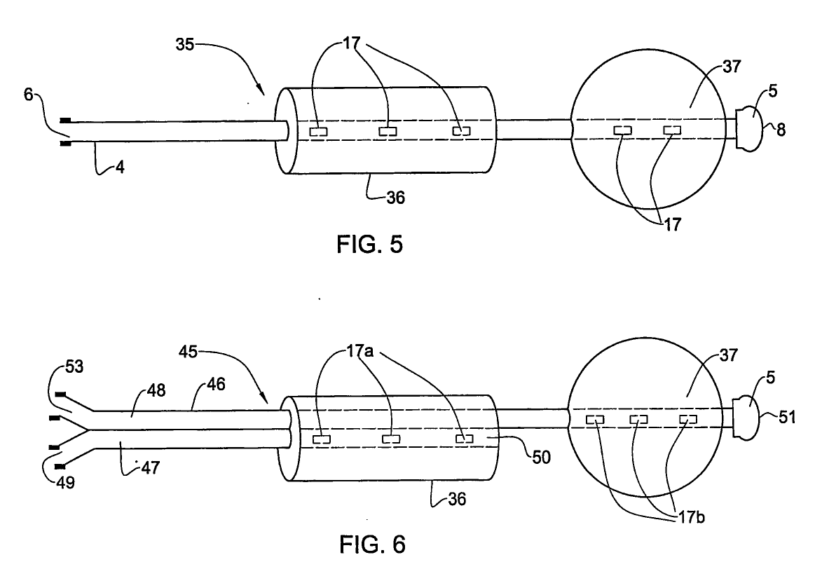

FIG. 6 shows another embodiment of the device of the invention, generally indicated by 45. The device 45 has features in common with the device 35, and similar components are identified with the same numeral without further comment. In particular, the embodiment 45 has two balloons 36 and 37, that are shown in their inflated state in FIG. 6. The balloon 36 has a cylindrical shape when inflated, and has dimensions similar to those of the cylindrical portion 9 of the balloon 12. The balloon 37 is ellipsoidal in shape and has dimensions similar to those of the ellipsoidal portion 11 of the balloon 12. In contrast to the device 35, the device 45 has a shaft 46 containing a first lumen 47 and a second lumen 48. The lumen 47 has a proximal end 49 and a distal end 50 located in the cylindrical balloon 36. Fluid is delivered from the lumen 47 into the cylindrical balloon 36 through openings 17a in the shaft 46. The lumen 48 has a proximal end 53 and a distal end 51 located distal to the spherical balloon 36. Fluid is delivered from the lumen 48 into the spherical balloon 37 through openings 17b in the shaft 46. Each of the lumens 47 and 48 may be connected to a pump 18 and reservoir 20 (as shown in FIGS. 2 to 4) so that the balloons 36 and 37 may be inflated independently. The device 45 is used similarly to the device 2 as shown in FIGS. 2 to 4. In particular, the ellipsoidal balloon 37 can be inflated before the cylindrical balloon 36. This is done in order to position the cylindrical balloon 36 in the cervix as described above with reference to the embodiment of FIG. 5.

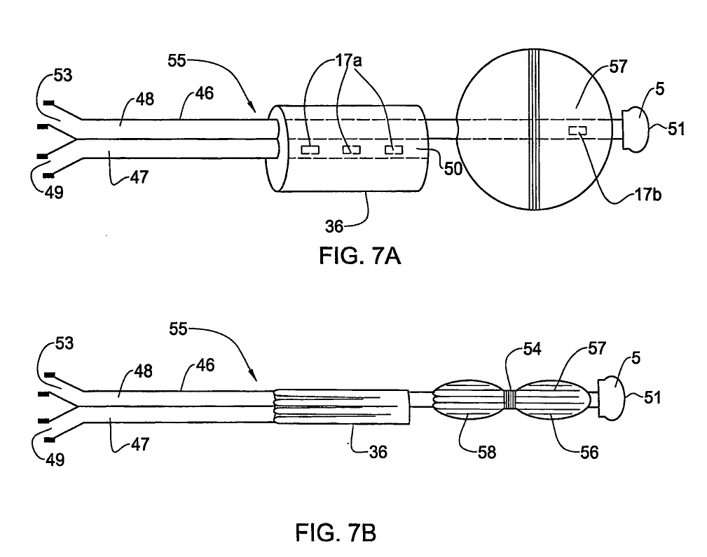

FIG. 7 shows another embodiment of the device of the invention, generally indicated by 55. The device 55 has features in common with the device 45 of FIG. 6, and similar components are identified with the same numeral without further comment. The embodiment 55 has two balloons 36 and 57, that are shown in their inflated state in FIG. 7a, and in their uninflated state in FIG. 7b. The balloon 36 has a cylindrical shape when inflated, and has dimensions similar to those of the cylindrical portion 9 of the balloon 12. The balloon 57 is ellipsoidal in shape when inflated and has inflated dimensions similar to those of the ellipsoidal portion 11 of the balloon 12. The balloon 57 when uninflated is constricted around the shaft 46, at an elastic constriction site 54 so as to form two separately inflatable compartments in the uninflated balloon 57. As explained in detail below, pressurized fluid in the lumen 48 is released only into a distal compartment 56. When fluid pressure in the distal compartment exceeds a predetermined value, the fluid forces passes from the distal compartment 56 into a proximal compartment 58, as explained in detail below.

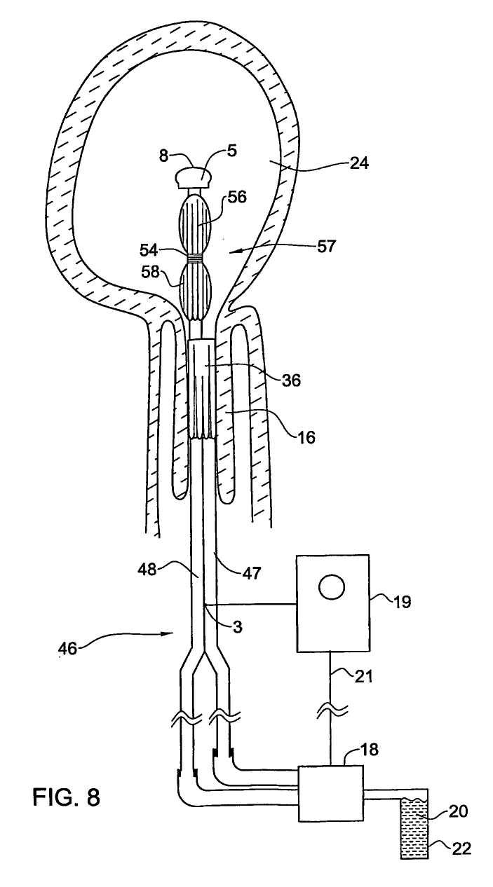

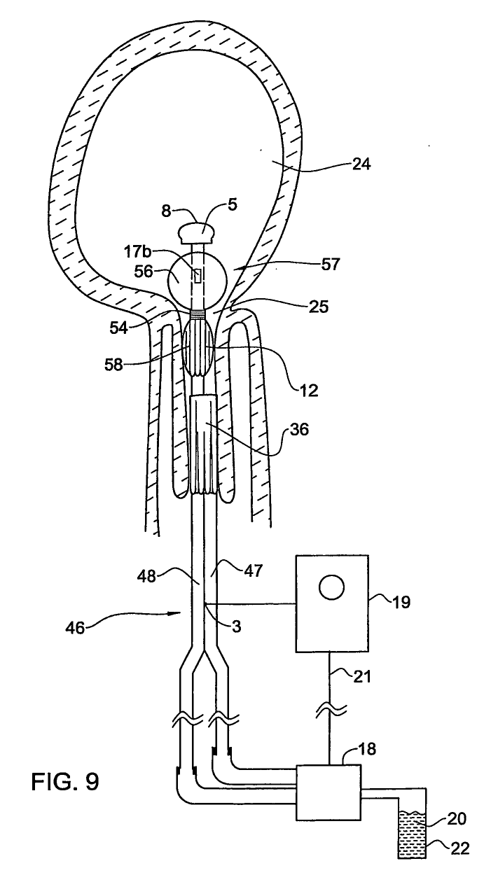

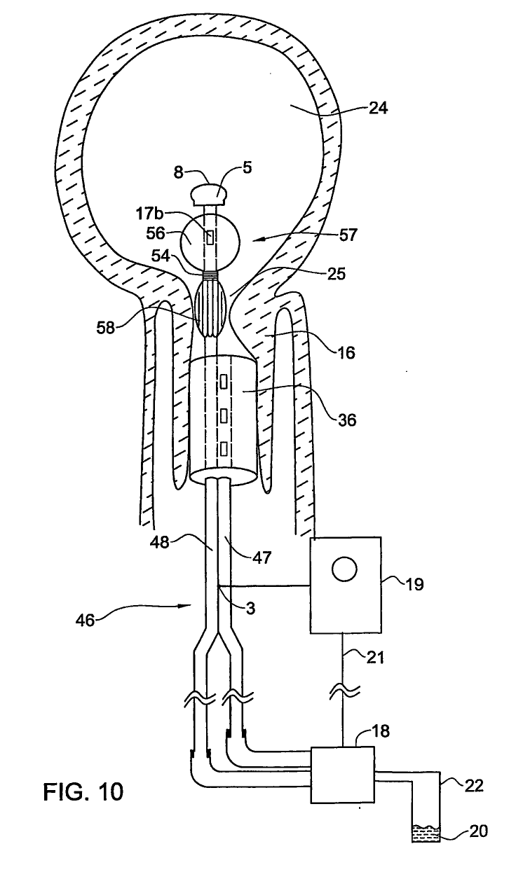

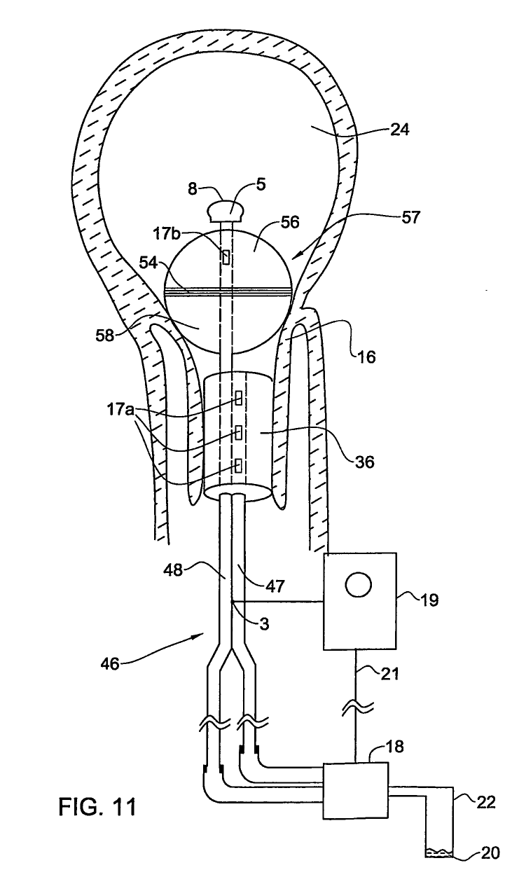

FIGS. 8 to 10 show deployment of the device 55. As shown in FIG. 8, the balloon 36 is delivered to the cervix 16 and the balloon 57 is delivered to the uterus 24, with the balloons 36 and 57 in their uninflated state and collapsed on the shaft 46. Fluid is now delivered through the lumen 48 of the shaft 46 and the opening 17b into the distal compartment 56 of the balloon 57, until inflation of the distal compartment 56 is complete, as shown in FIG. 9. Due to the elastic constriction area 54, the proximal compartment 58 of the balloon 57 does not inflate at this point. The inflated distal compartment 56 is then lodged in the internal os 25, so as to ensure that the cylindrical balloon 36 is properly positioned in the cervix 16. The proximal compartment 58 is now located in the distal termination of the cervix 16. Now pressurized fluid is delivered to the cylindrical balloon via the lumen 48 of the shaft 46. FIG. 10 shows the device 55 after inflation of the cylindrical balloon 36 is complete. Now additional fluid is delivered to the distal compartment 56 of the ellipsoidal balloon. When the fluid pressure in the distal compartment 56 exceeds a predetermined threshold, the elastic constriction area 54 expands allowing fluid to flow into the proximal compartment FIG. 11 shows the device with the balloon 57 completely inflated. As the proximal compartment expands 58, it causes the distal terminal segment of the cervix 16 to expand. A spherical balloon can withstand a pressure that is twice that of a cylindrical balloon made of the same material and having the same wall thickness. Thus, the spherical balloon can deliver a pressure to the distal terminal portion of the cervix (where the resistance to dilation is greatest) that is about twice the pressure that can be delivered to the cervix by the cylindrical balloon

1. A device for dilating a body passageway comprising:
(a) an elongated hollow shaft having a lumen, a proximal end and a distal end, and
(b) a balloon at the distal end of the shaft in fluid communication with the lumen of the shaft, characterized in that:
the balloon has a cylindrical portion and an ellipsoidal portion distal to the cylindrical portion.

2. The device according to claim 1 wherein the balloon is formed from polyethylene perephthalate (PET) or polyamide.

3. The device according to claim 1 or 2 flier comprising a reservoir for containing a fluid.

4. The device according to claim 3 wherein the reservoir contains an incompressible fluid.

5. The device according to claim 3 further comprising a pump for delivering fluid from the reservoir to the balloon.

6. The device according to claim 5 wherein the pump is configured to deliver fluid from the reservoir to the balloon at a predetermined or selectable flow rate.

7. The device according to claim 5 further comprising a pressure gauge measuring fluid pressure in the balloon and wherein the pump is configured to deliver fluid from the reservoir to the balloon so as to achieve a predetermined or selectable rate of increase of pressure in the balloon.

8. The device according to claim 5 wherein the pump has a flow rate that is remotely controlled.

9. The device according to any one of the previous claims wherein the balloon contains an area f is elastically constricted onto the shaft when the balloon is uninflated so as to form a distal compartment and a proximal compartment, the distal compartment being in fluid communication with the second lumen and the proximal compartment not being in fluid communication with the second lumen when fluid pressure in the distal compartment is below a predetermined threshold.

10. A device according to any one of the previous claims wherein the body passageway is a cervix, urethra, or blood vessel.

11. A method for dilating a body passageway comprising inserting the cylindrical portion of the balloon of a dice according to any one of the previous claims into the passageway and inflating the balloon.

12. The method according to claim 11 wherein the passageway is a cervix, urethra, or blood vessel.

13. The method according to claim 12 wherein the passage way is a cervix communicating with a uterus, and the device comprises a balloon having an ellipsoidal portion distal to the cylindrical portion, and the method comprises inflating the ellipsoidal portion in the uterus followed by inflating the cylindrical portion in the cervix.

14. The method according to claim 12 wherein the passage way is a cervix communicating with a uterus, and the device comprises a spherical or ellipsoidal balloon dial to the cylindrical portion, and the method comprises inflating the spherical or ellipsoidal balloon in the uterus followed by inflating the cylindrical portion in the cervix.

15. The method according to any one of claims 11 to 14 wherein one or more of the balloons is inflated with an in incompressible fluid.
